(19) United States
(12) Patent Application Publication
(10) Pub. No.: US 2003/0064509 A1
(43) Pub. Date: Apr. 3, 2003

(54) Novel chromosomal vectors and uses thereof

(76) Inventors: Peter Marynen, Herent (BE); Joris Vermeesch, Veltem-Beisem (BE); Thierry Voet, Heverlee (BE)

Correspondence Address: TRASK BRITT, P.O. BOX 2550, SALT LAKE CITY, UT, 84110, US

(21) Appl. No.: 10/251,008

(22) Filed: Sep. 20, 2002

Related U.S. Application Data
(63) Continuation of application No. PCT/EP01/03899, filed on Apr. 3, 2001.

(30) Foreign Application Priority Data
Apr. 7, 2000 (EP) 00201263.1

(51) Int. Cl. A61K 48/00; C12N 15/00

(52) U.S. Cl. 435/320.1; 514/44

(57) ABSTRACT

Novel chromosomal vectors, particularly human artificial chromosomes, which are efficiently transmitted through the male and female germ line in each generation. The vectors are also transmitted through mitosis in substantially all dividing cells and provide a position independent expression of an exogenous DNA sequence. These vectors can be used in gene therapy and are useful for the production of transgenic animals and plants and products thereof.

### CROSS-REFERENCE TO RELATED APPLICATION

This application is a continuation of International Application Number PCT/EPO1/03899 filed on Apr. 3, 2001, designating the United States of America, International Publication No. WO 01/77357 (Oct. 18, 2001), published in English, the contents of the entirety of which are incorporated by this reference.

### TECHNICAL FIELD

The present invention relates to novel chromosomal vectors, in particular to human artificial chromosomes, which are efficiently transmitted through the male and female germ line in each generation. The vectors are also transmitted through mitosis in substantially all dividing cells and provide a position independent expression of an exogenous DNA sequence. These vectors can be used in gene therapy, and are useful for the production of transgenic animals and plants and products thereof.

### BACKGROUND

As a result of the human genome project, the nucleic acid sequence of the entire human genome has become available. The identification of every gene in the human genome will provide insight into the mechanisms responsible for many diseases. However, a structural description of the human genome is not likely to be sufficient to allow an understanding of the mechanisms of gene regulation, which can depend on DNA regulatory elements that are located thousands of base pairs or more from the regulated gene. In addition, some genes, such as the dystrophin gene, contain over one million base pairs and, therefore, are too large to be conveniently transferred from one cell into another using currently available technology.

Stable transgenic eukaryotic cells (such as mammalian and plant cells) are currently essentially generated by random integration of foreign DNA into the host genome. This introduction of foreign DNA can mutate the host genome: the transgene can modify the properties of neighbouring host genes while the host genome itself can influence transgene expression^{1,2}. In addition, often more than one copy of the transgene is introduced in the host genome^{3,4} and insertion of foreign DNA can even lead to rearrangements and deletions^{5,6}.

Currently available mammalian vectors (such as retroviral vectors) can harbor DNA fragments with a maximum insert size of 10,000 nucleotides. In comparison, yeast artificial chromosomes (YACs) can harbor DNA fragments having a few hundred thousand nucleotides. However, YAC vectors are not stable in mammalian cells, unless inserted in the host-cell genome, and therefore are unsuitable to be used, for example, as vectors for gene therapy.

Different strategies have been followed to generate mammalian artificial chromosomes (MACs). In the “bottom up” approach, artificial chromosomes are generated de novo. In vivo self-assembled MACs were obtained after the introduction of human alphoid repeats in the human HT 1080 cell line together with total human genomic DNA and telomeric repeats⁹. Other groups generated de novo chromosomes by the introduction of yeast artificial chromosomes carrying centromeric alphoid repeats capped with chimerical yeast-human telomeric repeats in human HT 1080 cells^{10,11}. In both cases, the resulting de novo minichromosomes are estimated to be 2-10 megabases in size which is likely to be the result of a multimerization of the input sequences. MACs for making transgenic animals are also described in PCT International Publication WO 97/16533 to I. Scheffler. It should be noted, however, that no germline transmission of MACs has been observed!

In the “top down” approach, non-essential chromosomes present in somatic cell hybrids are reduced in size either by telomere-associated chromosome fragmentation (TACF)^{12,15} or by irradiation microcell-mediated chromosome transfer^{16,18}. Minichromosomes, all containing alpha satellite repeats, of less than 2.5 Mb have thus been created. Several authors also explored the possibility of using naturally occurring minichromosomes^{19,20}. Some examples of top down approaches are: PCT International Publication WO 95/32297 to W. Brown describing fragments derived from the human Y chromosome which can be used as vectors; European Patent Application EP/0838526 to J. Xia disclosing human satellite microchromosomes as vectors for gene therapy; and, PCT International Publication WO 00/18941 to H. Cooke et al. indicating that a mammalian artificial chromosome containing a mammalian centromere that comprises alphoid DNA replicates autonomously. However, in none of these documents, has an efficient germline transmission been shown. Also Hernandez et al. (1999)¹⁷ reported that in none of 41 male chimeras, generated using ES cells carrying a human chromosome 21-derived minichromosome, has germline transmission been observed. Shen et al. (2000)⁴⁰ reported the germline transmission of a human/mouse microchromosome only by the female chimeras. The same authors³⁹ further reported the germline transmission of this human/mouse microchromosome by one out of three female chimeras and none of six male chimeras. Also in F1 and F2 offspring not a single male germline transmission could be observed. Only in the third generation, the report mentions an isolated case of an F3 male which succeeded to transfer a minichromosome to a restricted number of its offspring. It is clear that such an accidental male transmission is very inefficient for breeding larger transgenic animals (for example cows or goats).

A different strategy is based upon the rescue by microcell-mediated chromosome transfer (MMCT) of chromosome fragments generated by irradiation. Tomizuka et al. (1997, 2000)^{48,49} have constructed a library of human-mouse A9 monochromosomal hybrids containing human chromosome fragments derived from normal embryonic fibroblasts. The library comprises approximately 700 independent hybrid clones. These were used as a source of microcell donor cell lines for MMCT into mouse ES cells. In this way, they could demonstrate mitotic stability under nonselective conditions for a human chromosome 14 fragment in female mouse TT2F ES cells (mitotic loss rate of less than 0.1%). However, these data are based on only one clone. Another fragment of human chromosome 2 was found to be much less stable (3.2% mitotic loss) in the same ES cells when grown in the absence of selection.

Recently, also germline transmission of these human chromosome 2 and 14 fragments carrying respectively the human Igκ light and the human IgH heavy chain locus by male and female F1 and F2 mice was demonstrated⁴⁹. The human chromosome 14 fragment was transmitted with a mean efficiency of 33% and 36% in male and female mice, respectively. However, the efficiency of germline transmission by F1 and F2 male mice containing the human chromosome 2 fragment was approximately 9% and thus much lower when compared with the efficiency of germline transmission of the same minichromosome by female mice (23%). To evaluate the stability of both human chromosomal fragments in somatic cells of these mice, metaphase spreads of tail fibroblasts were prepared and examined for the presence of the fragments by FISH. It was shown that approximately 78% of the cells contained the human chromosome 14 fragment while only 30% contained the human chromosome 2 fragment. Although this approach of fragmenting human chromosomes may result in some germline transmission, it is clear that the latter approach is not fast and practical and that it can not be predicted if each fragment carrying the desired coding sequence will be transmitted through the germline. It is also important to note that the latter chromosomal fragments consist solely of endogenous DNA directly derived from the chromosome they are a fragment of and do not contain an exogenous DNA sequence!

In summary, it is clear that efficient and predictable germline transmission in each generation, especially male germline transmission, of artificial chromosomes comprising exogenous DNA has not been demonstrated.

Moreover, an urgent need exists for vectors that: (1) are mitotically stable without selection, (2) allow the integration of very large fragments of foreign/exogenous DNA at a well-defined locus, (3) allow the regulated and position independent, stable expression of genes present on the vector, (4) are transferable among different cell lines and (5), most importantly, show stable and efficient male and female germline transmission as an independent chromosome in transgenic animals and plants.

The present invention satisfies this need.

### SUMMARY OF THE INVENTION

The invention includes a non-integrating chromosomal vector that is mitotically stable without selection. The vector allows the integration of very large fragments of foreign/exogenous nucleic acids at a well defined locus. It also allows the regulated and position independent expression of genes present on the vector. It is transferable among different cell lines, and shows stable and efficient male and female germline transmission as an independent unit in transgenic animals and plants.

In particular, the present invention aims at providing a non-integrating vector that: a) is transmitted through the male gametogenesis in each subsequent generation, and/or b) is transmitted through mitosis in all, or almost all, cells and/or c) allows for position independent expression of exogenous DNA. The invention further aims at providing a vector which has a transmittal efficiency through the male and female gametogenesis of at least 10%, preferably of at least 50%, more preferably of at least 75% and most preferably of 100%.

More particularly, the present invention aims at providing a, preferably circular, chromosomal artificial vector which efficiently passes through the male and female germ line of animals, in particular mammals, or plants.

More particularly, the present invention aims at providing a human artificial chromosome derived from a human small accessory chromosome having the above-described characteristics.

The present invention also aims at providing a method to produce such vectors and aims at providing particular uses of such vectors. The latter uses include, but are not limited to, the usage of the vectors for gene therapy in humans, for the production of non-human transgenic plants and animals, and for the production of recombinant proteins and secondary metabolites in cell culture.

### BRIEF DESCRIPTION OF DRAWINGS

FIG. 1 illustrates modification and characterization of the small accessory chromosome (SAC) structure of the different vectors and strategy for introduction of new sequences into the SAC by Cre-mediated recombination. SAC sequences are indicated with a thick black line, vector sequences with a thin black line, loxP sequences with a wide arrowhead. Neo: neomycine resistance gene driven by a thymidine kinase promoter, hyg: hygromycin resistance cassette driven by the PGK promoter, 5′- and 3′HPRT: human HPRT minigene driven by the SV40 early promoter. P: PstI cleavage site, B: BamHI cleavage site. Fragments used as a probe for Southern hybridizations are indicated with a double arrow (⇄). Not drawn to scale. HCV stands for Human Chromosomal Vector and is identical, as used herein, to HAC which stands for human artificial chromosome.

FIG. 2 shows tissue distribution of the Human Chromosomal Vector (HCV) and southern analysis of the HCV. DNA prepared from different tissues of an HCV⁺ F1 mouse was digested with XbaI, size-separated and blotted. The left panel shows the hybridization with a human alphoid-2 probe. The signal obtained for the different tissues is identical to the signal obtained for the E10B1 clone. The right panel shows the ethidium bromide stained agarose gels.

FIG. 3 illustrates how RNA was isolated from different tissues of a male (I) and a female (II) HCV⁺ F1 or HCV⁺ F2 mouse and brain of a normal control mouse. RT-PCR assays were developed detecting specifically human or mouse TF mRNA. Equal amounts of cDNA were used for 30 cycles of PCR with the human TF primers (hTF panels) or with the mouse F3 primers (mTF panels). A human fetal brain control is shown in lane C₁, lane C₂ shows a normal mouse brain control. A: RT-PCR experiments with cDNA derived from tissues of transchromosomal F1 and F2 mice. b: brain, k: kidney, l: liver, i: intestine, m: muscle, n: negative control. B: Western blot with 25 μg of total kidney proteins extracted from human kidney (hu), kidneys of 4 transchromosomal mice (respectively F1 I, F1 II, F2 I and F2 II) and a normal mouse (m), stained with rabbit anti human F3. C: Immunostaining with rabbit anti human TF of kidney, from a HCV⁺ (=HCV⁺) F1 mouse and a HCV⁻ littermate as a control, shows positivity of the epithelia of the glomerulus, a typical human expression pattern, only in the HCV⁺ (=HCV⁺) kidney. The top panel shows staining of a human control kidney section.

### DETAILED DESCRIPTION OF THE INVENTION

The present invention relates to non-integrating chromosomal vectors comprising an exogenous nucleic acid sequence that:

a) are transmitted through the male gametogenesis in each generation, and/or

b) are transmitted through mitosis in all, or almost all, dividing cells, and/or

c) allow for a position independent expression of an exogenous DNA sequence.

The present invention further relates to vectors that are efficiently transmitted through the female and male gametogenesis. The term “vector” refers to any nucleic acid capable of carring inserted foreign or exogenous nucleic acid, such as DNA, into a host cell for the purpose of producing a polypeptide or a protein encoded by the foreign DNA in said host cell or encoding a ribozyme or being able to generate an antisense fragment of an existing gene. The vector can be obtained by any method known to a person skilled in the art, such as the methods described in U.S. Pat. No. 6,025,155 to Hadlaczky et al. The term “chromosomal” refers to a vector carrying a centromere. The term “non-integrating” refers to vectors which do not insert into the genome of the host cell. The terms “female and male gametogenesis” refer to the production of gametes or mature germ cells. The female gametogenesis results in eggs or ova and the male gametogenesis results in spermatozoa or sperm. Ova (or egg nuclei) and sperm (or sperm nuclei) contain half the number of chromosomes compared to most somatic cells or vegetative cells.

The terms “in each generation” indicate that a male transformant (such as a chimera) carrying the vector of the present invention in its cells will transmit the vector to at least 1 individual of its offspring (F1), (for chimera this is assessed in at least three independent litters because in each chimera the transformed ES cells will not contribute to germ cell formation), and that in turn, an individual of said offspring which carries said vector will transmit said vector to at least 1 individual of its offspring (F2) (for animals this can be assessed in one litter) and so on to at least F3 and F4.

The term “transmission in substantially all dividing cells” indicates that the vector is transmitted during each mitosis with a maximal loss in 1% of the mitotic events. Preferentially, there is no (0%), 0.1%, 0.2%, 0.3%, 0.4%, 0.5%, 0.6%, 0.7%, 0.8% or 0.9% loss of vector per mitosis.

The relatively low loss of vector also results in transformants carrying the vector in at least 70%, but preferably at least 75%, 85%, 95% or 100% of all their cells.

The term “provides for a position independent expression of an exogenous DNA sequence” indicates that the vector of the present invention expresses exogenous (i.e., foreign) DNA sequence in tissue(s) of the transformant in a genuine way as to the tissues where said DNA is expressed in the organisms from which said (exogenous) DNA sequence is derived. In a “genuine way” means that the regulatory sequences of the exogenous DNA sequence control the expression of the gene or genes present on said DNA fragment in exactly the same way, for example in space and time, as in the organism from where this exogenous DNA fragment is derived.

The present invention relates, in particular, to vectors that have a transmittal efficiency through the male and female gametogenesis in animals or plants of, on average, at least 10%. The latter terms indicate that, on average, at least 10% of offspring from parents carrying the vector contain the vector. In this regard, it should be clear that during meiosis or gametogenesis homologous chromosomes pair to form a bivalent. Each chromosome of said bivalent will then be pulled to either pole of a cell so that the resulting gametes contain half the number of chromosomes. This means in theory that in case only one vector is present per germ cell (i.e., has no homologue), only 50% of the gametes will contain the vector so that on average 50% (if only 1 parent carries said vector) or 75% (if both parents carry said vector) of offspring carry said vector. However, an average of 50 to 75% of positive offspring does not exclude that a 100% positive offspring is still a possible outcome. It should therefore, and because multiple and/or homologous vectors can also be present per germ cell, be clear that the term “efficiency” as used herein is measured by determining the percentage of offspring carrying said vector and that said efficiency is preferably higher than 25% and can be 30%, 40%, 50%, 60%, 70%, 75%, 80%, 85%, 90%, 95%, 99% or 100%.

The present invention further concerns the efficient transmission of the above-indicated vectors through the gametogenesis occurring in animals and plants.

The term “animal” refers to any animal producing haploid germ cells and refers, in particular, to birds such as chickens and mammals such as mice, rats, rabbits, cows, pigs, goats, sheep, horses, primates and humans. The term “plant” refers to any plant, dicotyledons and monocotyledons, which produces egg nuclei and sperm nuclei in a pollen grain.

The present invention provides, in particular, a non-integrating human artificial chromosomal vector (HCV) according to the invention, comprising a functional centromere, a selectable marker and a unique cloning site. The invention also provides methods of using a HCV. For example, the invention provides methods of stably expressing a nucleic acid molecule in different cellular genomic backgrounds, comprising introducing a HCV containing the exogenous nucleic acid molecule into the cell. The invention also provides a method for generating a transgenic animal or plant carrying a recombinant HCV. This modified human artificial chromosome thus shows the properties of a useful chromosomal vector: it segregates stably as an independent chromosome, sequences can be inserted in a controlled way and are expressed from the vector, the HCV has some unique properties since it is efficiently transmitted through the male and female germline in mice and the transgenic mice bear the chromosome in >70% of the cells in essentially all tissues tested.

The HCV of the invention is also mitotically stable in different genetic backgrounds which is an important aspect determining its experimental usefulness.

The present invention also provides a method to produce a vector according to the invention such as the HCV. The HCV was isolated from human fibroblasts in which it was mitotically stable. After transfer into hamster cells and introduction of the loxP site and a selectable marker the HCV maintained its mitotic stability, showing a loss of less than 0.25 percent per mitosis in the absence of selection. This can be explained by the presence of an active centromere. Several studies using linear human microchromosomes already showed that these segregated properly in human or hamster cells in the absence of selection^{9,10,13,19}. There is however some evidence that the copy number of smaller minichromosomes (2.4 Mb) is more variable in human and hamster cell lines¹⁵.

Another aspect of the invention is the stable segregation of the HCV in mouse male R1 embryonal stem cells, showing 1% or less loss per mitosis in 4 out of five ES clones tested. Shen et al. (1997)²³ introduced human minichromosomes derived by TACF of the Y into the CGR8 ES cell line. These minichromosomes were rapidly lost from the ES cells in the absence of selection suggesting that human centromeres function poorly in ES cells. Several other groups reported the introduction of human minichromosomes into ES cells^{17,25} but it is difficult to compare the mitotic stability of these minichromosomes to this of the HCV because either the chromosomes were kept under selective pressure²⁵ or the number of population 17 doublings analysed was much lower¹⁷.

Another aspect of the invention is the mitotic stability of the HCV in mouse liver, lung and white blood cells from F1 mice carrying the HCV. Those cells were shown to carry the HCV in more than 85% of the cells by interphase FISH. Furthermore, analysis of tail fibroblast metaphases showed that the HCV was present as an independent chromosome. These data are corroborated by Southern data that demonstrate the presence of equal amounts of HCV derived human alphoid sequences in all tissues tested. The HCV is also structurally stable as it did not acquire mouse sequences which could not be visualized by FISH. Furthermore, the inter-alu PCR pattern obtained with DNA from the F1 HCV⁺ generation was identical to the one obtained from the E10B1 hybrid. Taken together, these data demonstrate that the HCV was not rearranged.

Another embodiment of the invention is the efficient male and female germline transmission of the HCV. The high stability of the HCV in R1 ES cells suggested the possibility to use this chromosome to generate transchromosomal mice. Two normal male chimeric HCV⁺ mice were obtained and mated with female C57B1/6s mice to test the germline transmission of the extra human minichromosome. It was observed that the HCV was efficiently transmitted by both chimeras. In addition, both male and female F1 HCV⁺ mice efficiently transmitted the HCV to their offspring and the HCV in the mice seems very similar to the original HCV as was characterized in the hamster hybrid cell line. No particular phenotype was associated with the presence of the HCV in any of the HCV⁺ mice born. Thus, the HCV described in this invention is efficiently transmitted through both the male and female germline. This suggests that the HCV is not recognized as an unpaired chromosome during gametogenesis in the mouse. It is unlikely that this would be the result of the small size of the HCV. We have not been able to determine in an unambiguous way the size of the HCV as it does not migrate into PFGE gels nor could it be detected on Southern blots of PFGE experiments performed after irradiation of the plugs. The intensity of the DAPI staining however indicates that the HCV has about 20% of the size of the smallest human chromosome and it can thus be estimated at 5-10 Mb. This is well within the range of the other minichromosomes which have been generated. A major structural difference between the HCV and the artificial chromosomes reported by others^{10,17,25}, is the absence of detectable telomere repeats, suggesting that the HCV is a circular chromosome.

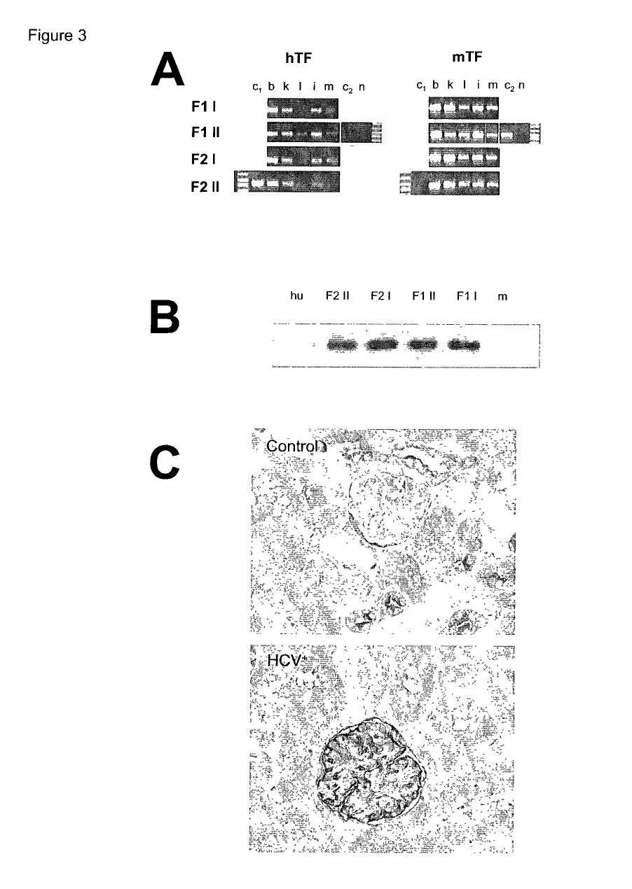

Another embodiment of the invention is the stable expression of genes present on the HCV. The generation of HPRT⁺ CH cells by reconstitution of a human HPRT minigene on the HCV shows that expression of genes present on the HCV occurs. The proportion of G418 fibroblasts derived from HCV⁺ F1 mice is similar to the proportion of HCV⁺ fibroblasts detected by FISH. This suggests that no extensive and strong position effect variegation does occur. Furthermore, the human tissue factor (TF) gene which is present on the HCV has a typical human expression pattern (FIG. 3). This demonstrates that the regulating sequences of the human TF gene are fully functional on the HCV and that the vector of the present invention allows for a position independent expression.

Another embodiment of the invention is that very large gene fragments can be introduced on the HCV via site-specific integration with the LoxP site present on the HCV. This Cre-recombinase mediated integration is only an example and other recombination mediated integration methods can be used.

Thus, artificial chromosomes such as HCV provide convenient and useful vectors, and in some instances (e.g., in the case of very large heterologous genes) the only vectors, for introduction of heterologous genes into hosts. Virtually any gene of interest is amenable to introduction into a host via artificial chromosomes. Such genes include, but are not limited to, genes that encode receptors, cytokines, enzymes, proteases, hormones, growth factors, antibodies, tumor suppressor genes, therapeutic products. This new vector could be particularly useful for the introduction of complete metabolic, which often consist of multiple genes under control of their own, natural or a different or regulated promoter. The latter application can be highly beneficial for the production of specific compounds of proteins in animal or plant cell culture. Together with the high mitotic stability of the HCV in cell cultures makes this new vector an attractive tool.

The artificial chromosomes provided herein can be used in methods of protein and gene product production of important compounds for medicine and industry. They are also intended for use in methods of gene therapy (ex vivo or in vivo) and for production of transgenic plants and animals.

Any nucleic acid encoding a therapeutic gene product or product of a multigene pathway may be introduced into a host animal, such as a human, or into a target cell line for introduction into an animal, for therapeutic purposes. Such therapeutic purposes include, gene therapy to cure or to provide gene products that are missing or defective, to deliver agents, such as anti-tumor agents, to targeted cells or to an animal, and to provide gene products that will confer resistance or reduce susceptibility to a pathogen or ameliorate symptoms of a disease or disorder. As used herein, gene therapy involves the transfer or insertion of heterologous DNA into certain cells, target cells, to produce specific gene products that are involved in correcting or modulating disease. The DNA is introduced into the selected target cells in a manner such that the heterologous DNA is expressed and a product encoded thereby is produced. Alternatively, the heterologous DNA may in some manner mediate expression of DNA that encodes the therapeutic product. It may encode a product, such as a peptide or RNA that in some manner mediates, directly or indirectly, expression of a therapeutic product.

Gene therapy may also be used to introduce therapeutic compounds that are not normally produced in the host or that are not produced in therapeutically effective amounts or at a therapeutically useful time. Expression of the heterologous DNA by the target cells within an organism afflicted with the disease thereby enables modulation of the disease. The heterologous DNA encoding the therapeutic product may be modified prior to introduction into the cells of the afflicted host in order to enhance or otherwise alter the product or expression thereof. As used herein, heterologous or foreign DNA and RNA are used interchangeably and refer to DNA or RNA that does not occur naturally as part of the genome in which it is present or which is found in a location or locations in the genome that differ from that in which it occurs in nature. It is DNA or RNA that is not endogenous to the cell and has been exogenously introduced into the cell. Examples of heterologous DNA include, but are not limited to, DNA that encodes a gene product or gene product(s) of interest, introduced for purposes of gene therapy or for production of an encoded protein. Other examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced. As used herein, a therapeutically effective product is a product that is encoded by heterologous DNA that, upon introduction of the DNA into a host, is expressed and effectively ameliorates or eliminates the symptoms, manifestations of an inherited or acquired disease or cures said disease.

The following are some exemplary genes and gene products. Such exemplification is not intended to be limiting.

a) Anti-HIV ribozymes DNA encoding anti-HIV ribozymes can be introduced and expressed in cells using HCVs. These HCVs can be used to make a transgenic mouse that expresses a ribozyme and, thus, serves as a model for testing the activity of such ribozymes or from which ribozyme-producing cell lines can be made. Such systems further demonstrate the viability of using any disease-specific ribozyme to treat or ameliorate a particular disease. Also, introduction of a HCV that encodes an anti-HIV ribozyme into human cells will serve as treatment for HIV infection. The introduction of foreign DNA in human hematopoietic stem/progenitor cells by micro-injection has been demonstrated (Davis et al. (2000))⁴¹, and could be adapted to introduce the HCV into these cells.

b) The CFTR gene Cystic fibrosis (CF) is an autosomal recessive disease that affects epithelia of the airways, sweat glands, pancreas, and other organs. It is a lethal genetic disease associated with a defect in chloride ion transport, and is caused by mutations in the gene coding for the cystic fibrosis transmembrane conductance regulator (CFTR), a 1480 amino acid protein that has been associated with the expression of chloride conductance in a variety of eukaryotic cell types. Defects in CFTR destroy or reduce the ability of epithelial cells in the airways, sweat glands, pancreas and other tissues to transport chloride ions in response to cAMP-mediated agonists and impair activation of apical membrane channels by cAMP-dependent protein kinase A (PKA). Given the high incidence and devastating nature of this disease, development of effective CF treatments is imperative. The CFTR gene (about 250 kb) can be transferred into a HCV for use, for example, in gene therapy. Mice carrying a CFTR-HCV can be used to investigate the spatial-temporal regulation of CFTR transcription. Therapy can be considered for tissues such as airway epithelia that are accessible, e.g. by liposomes that can be used as a delivery system for the CFTR-HCV.

Another embodiment of the use of artificial chromosomes in generating disease-resistant organisms involves the preparation of multivalent vaccines. Such vaccines include genes encoding multiple antigens that can be carried in a HCV, or species-specific artificial chromosome, and either delivered to a host to induce immunity, or into eukaryotic cell lines to produce the multivalent antigens.

Disease-resistant animals and plants may also be prepared in which resistance or decreased susceptibility to disease is conferred by introduction into the host organism or embryo of artificial chromosomes containing DNA encoding gene products (e.g., ribozymes, proteins that are toxic to certain pathogens, decoy receptors for pathogens or modified receptors that are no longer able to bind the pathogen) that destroy or attenuate pathogens or limit access of pathogens to the host.

Animals and plants possessing desired traits that might, for example, enhance utility, processibility and commercial value of the organisms in areas such as the agricultural and ornamental plant industries may also be generated using artificial chromosomes in the same manner as described above and further for production of disease-resistant animals and plants. In such instances, the artificial chromosomes that are introduced into the organism or embryo contain DNA encoding gene products that serve to confer the desired trait in the organism. As used herein, transgenic animals and plants refer to animals and plants in which heterologous or foreign DNA is expressed or in which the expression of a gene naturally present in the plant has been altered.

The following examples are included for illustrative purposes only and are not intended to limit the scope of the invention.

### EXAMPLES

### Example I

### Isolation, Modification and Characterization of a Human SAC

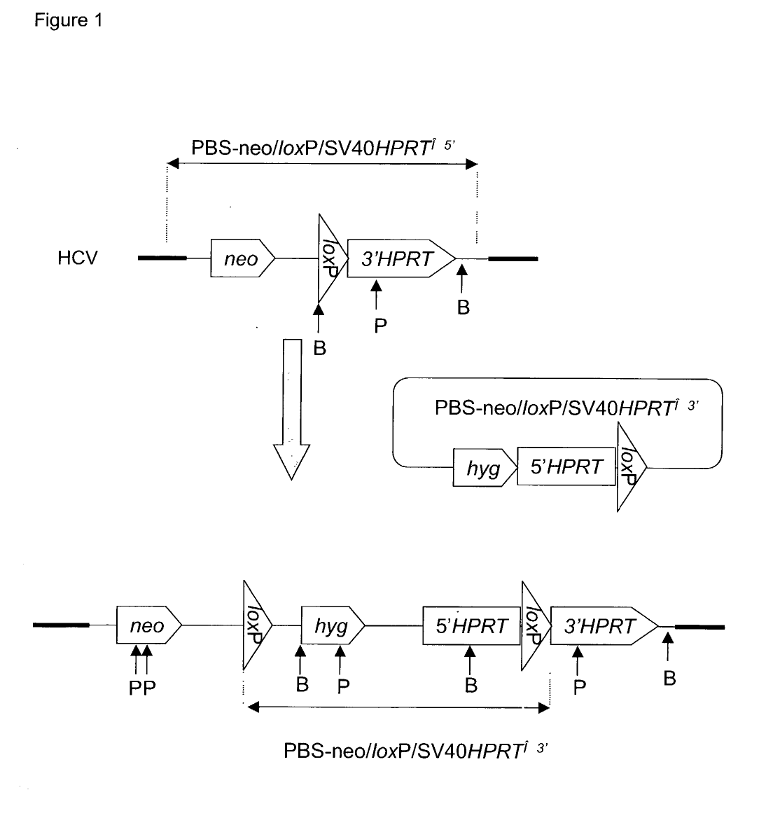

We previously characterized five mitotically stable SACs carrying a functional centromere as indicated by the presence of CENP-C proteins and without telomere sequences indicating that they were circular chromosomes²¹. Permanent cell lines containing the SACs were obtained by fusing these fibroblasts to hamster CH cells. To confer some properties of a vector to the SACs, the hybrids were transfected with the plasmid pBS-neo/loxP/HPRT^{-Δ5} (FIG. 1). This plasmid contains the neomycin resistance gene under control of a thymidine kinase promoter, followed by a loxP sequence and the 3′ end of a human HPRT minigene. The neomycin resistance gene allows the positive selection of somatic cell hybrids containing the SAC while the loxP/HPRT^{-Δ5} sequence provides a cloning site. The size of the diploid hamster genome is about 6000 Mb and from cytogenetics we estimated the size of the SACs to be 5-10 Mb, hence, assuming random integration, about 0.1% of the pBS-neo/loxP/HPRT^{-Δ5} molecules would be integrated into a SAC. To select for SACs with an integrated pBS-neo/loxP/HPRT^{-Δ5}, microcells were generated from the primary transfectants and size-selected²². The size fraction with the smallest microcells was subsequently fused with hprt⁻ CH cells. The resulting G418 resistant hamster/human somatic cell hybrids were screened for the presence of a SAC by PCR and FISH (not shown). A hybrid hamster cell line, E10B1, containing one human SAC (also referred to as Human Chromosomal Vector 1 (HCV1) was selected for further analysis and is deposited with the Belgian Coordinated Collections of Microorganisms-BCCM™ represented by the Laboratorium voor Moleculaire Biologie-Plasmidencollectie (LMBP), University of Ghent, K. L. Ledeganckstraat 35, B-9000 Ghent, Belgium on March 28, 2000 and has accession number LMBP 5473CB.

Lissamine-labeled human CotI and biotine-labelled pBS-neo/loxP/Hprt^{-Δ5′} cohybridized exclusively to the SAC in metaphase spreads of E10B1. The SAC is thus the only human chromosome present in this somatic cell hybrid and it is the only chromosome with an integrated pBS-neo/loxP/HPRT^{-Δ5′}.

The mitotic stability of the minichromosome in the hamster cell line was measured after 109 population doublings in the presence or absence of G418 (Table 1). FISH was performed on metaphase spreads to detect an eventual integration of SAC sequences into the hamster chromosomes. After 109 population doublings the mitotic loss of the SAC was less than 0.25% per mitosis in the absence of any selective pressure. Immunofluorescence using an anti-CENP-C antibody resulted in two bright spots on the SAC, showing that its stability is due to the presence of an active centromere. To test whether this might be due to integration of hamster centromeric DNA in the minichromosome we hybridized metaphases of E10B1 with hamster CotI DNA. No hybridization signals were present on the SAC while all hamster chromosomes were brightly stained.

The isolation of the SAC in a hamster cell line allowed us to investigate the human sequences present on the SAC in more detail. Inter-alu PCR detected a discrete number of human sequences on the SAC FISH with the inter-alu products of E10B1 on metaphase spreads of a HCV⁺ human cell line HT1080 showed signals on the HCV and unexpectedly on chromosome region 1p. Sequencing of a number of the inter-alu PCR products confirmed the 1p origin of the sequences and allowed us to detect the presence of the human tissue factor (TF) gene on the HCV.

Example II

### Recombination-Mediated Introduction of New Sequences in the SAC and Expression

To investigate the site-directed introduction of new sequences into the SAC we constructed a plasmid pBS-Hyg/SV40Hprt^{-Δ3′}/loxP containing a hygromycin resistance expression cassette followed by the 5′ end of the human hprt minigene controlled by the SV40 early promoter and a loxP sequence (FIG. 1). The hprt⁻ E10B1 hybrid was cotransfected with pOG231, a Cre-expression plasmid, and pBS-Hyg/SV40Hprt^{-Δ3′}/loxP. Homologous recombination at the loxP sequence would then reconstitute the human HPRT minigene (FIG. 1) and these clones can thus be selected in HAT medium. As negative controls E10B1 was mock-transfected or transfected with pBS-Hyg/SV40Hprt^{-Δ3′}/loxP or pOG231 only. No cells survived HAT selection in any of the negative controls. Over 200 resistant clones grew out of the cells cotransfected with pOG231 and pBS-Hyg/SV40Hprt^{-Δ3′}/loxP. This accounts for a recombination efficiency of 1.6×10⁻⁵/cell.

The correct reconstitution of the human HPRT minigene was demonstrated by PCR analysis on genomic DNA isolated from 10 clones with HPRT primers spanning the loxP site. The predicted 2.1 kb PCR product was obtained with genomic DNA from all clones but not with control genomic DNA derived from untransfected E10B1 cells (result not shown). In addition, the DNA from the clones was digested with either PstI or BamHI, size-fractionated by agarose gel electrophoresis, blotted and probed with a fragment of either the hygromycine resistance gene, the 5′ HPRT gene or the 3′ HPRT gene. Upon hybridization of the PstI digested DNA with the hygromycine probe, two signals of respectively 4 and 4.4 kb are expected. In five out of nine clones, this was indeed the case, while in the other clones some rearrangements and/or amplifications did occur. In two clones a signal was visible at 7.2 kb. When the same Southern blot was hybridized with the 5′ end of the HPRT gene, a signal at the same position was obtained. This suggests that the 7.2 kb band results from the integration of multiple copies of pBS-Hyg/SV40HPRT^{-Δ3′}/loxP at the loxP site. Upon hybridization, the BamHI digested DNA with the 3′ HPRT probe, a hybridization signal of 2.3 kb is expected before integration and a 3.5 kb hybridization signal upon correct integration. This 3.5 kb fragment is present in all clones. However, in 4 clones the original 2.3 kb fragment was also be present. We suggest that this might be due to a duplication of the circular SAC as a result of Cre-mediated sister chromatid exchange. Indeed a Cre-mediated homologous recombination occurring between the two loxP sites present on each sister chromatid of the circular SAC after DNA-replication could result in a duplication of the SAC. If subsequently, in one of the two sites an integration of plasmid pBS-Hyg/SV40Hprt^{-Δ3′}/loxP took place, both the original 2.3 and the rearranged 3.5 kB would be present. Taken together we conclude that 4 clones out of 10 (clones 1, 2, 7 and 10) had a correctly integrated plasmid pBS-Hyg/SV40HPRT^{-Δ3′}/loxP without obvious rearrangements.

To confirm that the HAT resistance of the clones was the result of the correct reconstitution and expression of the human HPRT minigene, RT-PCR was performed on RNA isolated from three clones. The amplified cDNA was of the correct size and subsequent sequencing of the RT-PCR product confirmed expression of the human HPRT minigene.

To investigate the site-specific integration of large genomic fragments into the HCV, a PAC clone containing the complete human CD4 gene (>90 kb) was isolated from the RPCI-6 library. The PAC vector (pPAC4) contains a eukaryotic blasticidin resistance expression cassette and a loxG site, compatible with the loxP site in the HCV for Cre-mediated recombination. The pPAC4-CD4 clone was used without modification and its DNA was cotransfected with the Cre expression plasmid pOG231 into the E10B1 cell line. FISH analysis showed that 1 out of 39 blasticidin resistant cell lines had integrated at least one copy of the pPAC4-CD4 clone into the HCV. PCR with primers designed to amplify the recombined lox sites demonstrated that the insertion occurred into the loxP site of the HCV.

The SAC thus shows a number of salient features of a chromosomal vector and is called a human chromosomal vector (HCV).

Example III

### Transfer of the HCV to Mouse ES Cells and Generation of Chimeras

Using microcell-mediated chromosome transfer (MMCT) the HCV was transferred into a male mouse ES cell line (R1). Nine G418 resistant hybrids containing the HCV were obtained, five of them were expanded and characterized.

The five hybrids were maintained with and without G418 selection for 40 population doublings and the presence of the HCV was investigated by FISH with labelled human Cot1 DNA (Table 1). Chromosome loss rates of the different ES clones in the absence of selection were low and varied between 2.66% and 0.26% per mitosis. FISH analysis using human Cot1 DNA as a probe confirmed the presence of the HCV as an independent chromosome in the ES cells. No FISH signal was visible on the HCV with either a mouse or hamster Cot1 probe indicating that little or no mouse or hamster DNA was integrated into the HCV. This experiment also showed that no hamster chromosomes were cotransferred to the ES cells. To determine whether an active centromere was present on the HCV, immunofluorescence was performed with anti-CENP-C and FISH with human CotI DNA on metaphase spreads of the ES cell hybrids. A CENP-C signal was visible on all mouse centromeres as well as, although fainter, on the HCV. Hence, the stable segregation of the HCV is due to the presence of an active centromere.

We injected male ES clone G or I cells into C57BL/6 blastocysts to create chimerical mice carrying the HCV. Two male chimeras (derived from clone G) and one female chimera (derived from clone I) were obtained from 3 independent experiments. PCR experiments on tail DNA of the chimeras using primers spanning the loxP site and primers derived for human 1p sequences present in the HCV (see below) were positive for the male chimeras and negative for the female chimera. FISH analysis performed on cultured tail fibroblasts from the male chimeras with a human CotI probe showed that the HCV was present in respectively 16 and 22% of the cells.

### Example IV

### Germline Transmission of the HCV in Mice

The two male chimeras were mated with female C57BL/6 and dominant-agouti offspring was obtained from both (Table 2). PCR detected the HCV in respectively 20 and 44% of the agouti offspring. FISH analysis on primary tail fibroblasts of 5 of the F1 transchromosomal mice indicated that the HCV was still present as an independent human chromosome among the 40 normal mouse chromosomes. No signal could be detected on the HCV by FISH using mouse CotI as a probe indicating that little or no mouse DNA was integrated into the HCV. In each of the 5 transchromosomal mice tested, about 85% of the nuclei from the tail fibroblasts contained a single HCV, the remaining nuclei showing no signal. No nuclei were observed with two or more signals. Simultaneous immunofluorescence staining with anti-CENP-C and FISH with a centromere 2 alphoid probe detected a CENP-C signal on both kinetochores of the HCV. FISH with a peptide nucleic acid telomere probe showed that the HCV contained no telomeric sequences. Taken together, this indicates that germline transmission did not change major functional properties of the HCV.

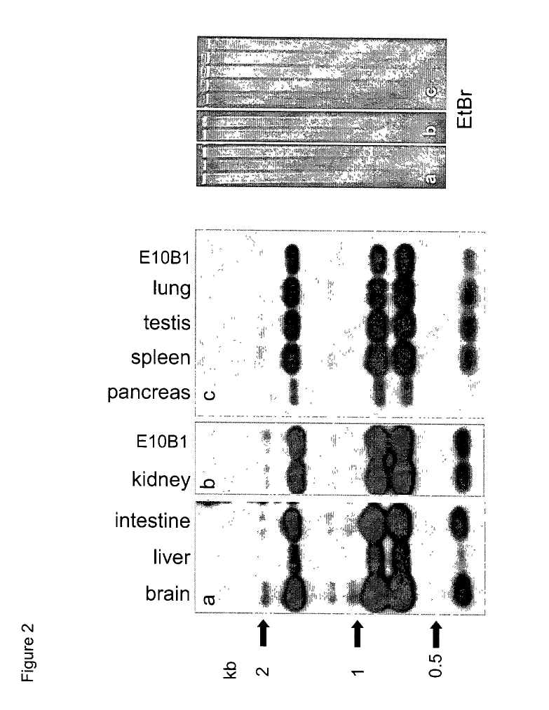

To investigate the tissue distribution of the HCV in the F1 mice, a HCV⁺ mouse was sacrificed and DNA was isolated from different tissues. A Southern with XbaI digested DNA was then hybridized with a human alphoid 2 probe (FIG. 2). DNA of the E10B1 HCV⁺ cell line was included as a control. Identical signals were obtained for all tissues tested and the hamster hybrid showing that the HCV was present in all mouse tissues with a similar copy number. Interphase FISH using a human alphoid 2 probe on liver, lung and white blood cells of 2 HCV⁺ F1 mice was in agreement with the Southern results (not shown). The presence of human sequences in the HCV⁺ F1 mice was also investigated. Inter-alu PCR with tail DNA of 5 HCV⁺ F1 mice generated a discrete number of products indistinguishable from those obtained with DNA of E10B1. Taken together with the Southern data this shows that male germline transmission of the HCV did not result in gross rearrangements of the HCV.

Next male and female F1 HCV⁺ mice were mated with C57BL/6s mice of the opposite sex. F2 HCV⁺ mice were identified by PCR with 1p primers on genomic tail DNA (Table 2). Both male and female transchromosomal mice showed efficient germline transmission of the HCV. FISH analysis with a centromere 2 alphoid probe and a mouse CotI probe confirmed these results.

### Example V

### Expression of HCV Genes in Transchromosomal Mice

Tail fibroblasts of the F1 HCV⁺ mice did proliferate in medium containing 800 μg/ml G418 whereas fibroblasts of HCV⁻ F1 agouti offspring died rapidly in this medium, demonstrating expression of the neomycin resistance gene from the HCV. When an equal amount of tail fibroblasts of two transchromosomal mice was seeded in medium with or without G418 respectively 91% (100 G418-resistant colonies against 110 in the control) and 83% (96/115) of the cells were G418 resistant. This is consistent with the number of HCV⁺ cells as detected by FISH, suggesting that all HCV⁺ cells do express the neomycin gene.

The presence of the human TF gene on the HCV provided an opportunity to evaluate expression of a human gene driven by its own promoter in the HCV⁺ mice. cDNA was synthesized form different tissues of a HCV⁺ F1 mouse and used as a template for PCR reactions with primer sets selectively amplifying the human or the mouse TF gene. FIG. 3A shows that the expression of human TF mRNA is variable in different mouse tissues, but that the expression levels are very similar in different transchromosomal animals of two generations. The highest expression was observed in the brain, kidney and intestine, low expression was seen in muscle, while very little human TF mRNA could be detected in liver. A Western blot stained with rabbit anti human TF detects similar amounts of TF in kidney samples of 4 transchromosomal mice with an Mr identical to the one observed for a human kidney sample (FIG. 3B). When the expression of TF in kidney was analysed by immunostaining of tissue sections, the epithelia of the glomeruli and some tubuli of HCV⁺ animals were clearly positive, whereas in HCV⁻ kidneys the glomeruli were negative (FIG. 3C). As shown by Luther et al., this is the typical human expression pattern of TF in kidney²² demonstrating the functionality of the regulatory sequences of the human TF gene on the HCV.

### Example VI

### Germline Transmission and Gene Expression in Plants

The novel HCV could also be used for the generation of transgenic plants. In a typical experiment protoplasts of the model plant Aribidopsis thaliana are prepared and are fused with donor cells containing the HCV via microcell-mediated chromosome transfer. Alternatively the plant protoplasts can also be microinjected with a pure preparation of the HCV. Selection for the plant protoplasts containing the HCV can be done in the appropriate medium depending on the selection marker present on the HCV, for example the antibiotic G418. Transformed protoplasts can be grown to callus tissue and this can be regenerated efficiently into mature recombinant plants. A functional plant chromosomal vector can be used for the generation of stable transgenic plants that can propagate the desired traits into their seeds. Since the novel vector can host large inserts of DNA wishful traits such as a collection of a wide variety of pathogen disease resistance genes and novel biochemical pathways can be transferred to plants.

### Example VII

### Further Characterization of the HCV

VII A Meiotic Stability

Additional experiments were performed aimed at the analysis of germline transmission by male and female mice. In the female meiosis (129SV×C57B16; 9 consecutive litters analysed), the HCV is passed to the progeny with an average efficiency of 36% (Table 3). The female HCV⁺ F1 mice were mated with the C57B1/6 strain and normal litter sizes were observed. With the male HCV⁺ F1 mice mated with C57B16 a slightly lower germline transmission of 31% was observed. However, small litter sizes of 1 to a maximum of 10 pups were produced. To analyze whether this was the result of subfertility of the males, or of independent factors, the same males were mated to the NMRI strain. In this case normal litter sizes were obtained. Litter sizes are thus strain dependent and are not due to HCV properties. The germline transmission efficiency in the 129SV×C57B16×NMRI crosses was 27%.

VII B Boundary of Human Chromosome 1p22 Insert

Sequencing of a number of the inter-ALU PCR products, using E10B1 genomic DNA as template, allowed us to generate primers for three STSs. YACs containing one or more of the three generated STSs were identified by screening the megaYAC library. All YACs contained fragments of human chromosome 1p22. Thirteen STSs mapping to human chromosome 1p22 were then tested on E10B1 genomic DNA by PCR. The proximal boundary of the 1p fragment on the HCV is located between D1S2868 (absent on the HCV) and WI-9122 (present). The distal boundary is located between WI-1974 (present on the HCV) and WI-7967 (absent). All STSs tested derived from the 1cM-2cM region bordered by WI-9122 and WI-1974 were present in the HCV.

VII C Vector Properties

a) Integration of PACs

To study the site-specific integration of large sequence fragments in the HCV and to analyze the tissue and time specific gene expression from the HCV, PACs based on the pPAC4 vector (containing a loxP site and a mammalian blasticidin selectable marker) and carrying the human CD4 or β-casein gene were isolated. The use of pPAC4 clones represents a simplification of the model compared to the insertion of plasmids. In this case, no selection occurs of the correctly inserted PACs as these clones do not contain the 5′-HPRT minigene cassette able to complement the 3′-HPRT minigene cassette present on the HCV. As the insertion of the PAC into the loxP site is a reversible process, and there is no selection against insertion of the PAC in the host genome, correct insertion into the HCV is expected to be less efficient.

Experiments were performed by co-transfecting 12.5.10⁶ E10B1 cells with 25 μg PAC DNA and 18.75 μg CRE expression plasmid DNA and selecting with G418 and blasticidin. In each experiment clones containing the HCV with the PAC inserted in a loxP site were obtained. Each experiment generated at least 30 clones.

In the case of the CD4 gene 99 clones were analyzed by a PCR detecting loxP integration. Three positive clones were identified. FISH using the PAC as a probe together with a HCV specific probe showed that the HCV with insert was consistently integrated into a hamster chromosome in two of these clones. The third clone was heterogeneous containing cells with a normal HCV containing a PAC insert, cells with amplified HCV sequences together with or without amplified PAC insert, and rare cells were the HCV and PAC were integrated in a hamster chromosome. Microcells generated from this clone were fused to the mouse ES-R1 cell line. Sixteen out of 62 G418 resistant clones contained a HCV with PAC insert. This included one clone with a normally sized HCV with a single PAC insert.

In the case of the PAC containing the human β-casein gene, 200 selected clones were analyzed for site-specific insertion of the PAC by a specific PCR. Eight clones were found to be positive and also contained heterogeneously sized HCVs by FISH as described above, but integrations of the HCV in hamster chromosomes were never observed. Sub cloning one of the 8 selected clones resulted in 3 sub clones (of the 8 analyzed) containing normally sized HCVs with a single PAC insert.

From the integration experiments with both PACs it can be concluded that the insertion of genomic PAC clones into the loxP site is possible, without specific selection of the correct insertion events. In both cases we observed amplifications of either the HCV sequences, the PAC sequences or both. In both cases it was possible to isolate clones with a HCV of a size similar to the original one and the insertion of one PAC.

The results suggest that the amplification of the HCV sequences and or of the PAC sequences, also observed during these experiments are not dependent on the integrated sequence (different PACs give similar results) or on the hamster cell line (equally sized HCVs can be obtained by sub cloning). Integration dependent amplifications appears to be due to the current location of the loxP integration site. This property can be used for the generation of HCV with multiple copies of one insert.

b) Gene Expression and Position Effects

Tail fibroblasts of the F1 and F2 HCV⁺ mice did proliferate in medium containing 800 μg/ml G418, whereas fibroblasts of HCV⁻0 F1 and F2 mice died rapidly in this medium, showing expression of the neomycin resistance gene from the HCV. When equal numbers of tail fibroblasts are seeded in medium with or without G418, the amount of clones growing in G418 is similar to the amount of HCV⁺ fibroblasts as detected by FISH in the cultures without G418 (Table 4). This suggests that all HCV⁺ cells do express the neomycin gene and little or no position effects disturb its expression.

TABLE 1
Mitotic stability of the HCV

| | | |
|---|---|---|
| population | +G418 | −G418 |

| Cell line | doublings | 0 | 1 | >1 | 0 | 1 | 2 |
|---|---|---|---|---|---|---|---|
| E10B1 | 109 | 1 (0, 9%) | 106 (95.5%) | 4 (3.6% | 19 (14.4%) | 103 (78%) | 10 (7.5%) |
| ES clone E | 40 | 2 (4%) | 48 (96%) | 0 | 33 (66%) | 17 (34%) | 0 |
| ES clone F | 40 | 0 | 50 (100%) | 0 | 17 (34%) | 33 (66%) | 0 |
| ES clone G | 40 | 3 (6%) | 47 (94%) | 0 | 5 (10%) | 45 (90%) | 0 |
| ES clone H | 40 | 1 (2%) | 49 (98%) | 0 | 11 (22%) | 39 (78%) | 0 |
| ES clone I | 40 | 1 (2%) | 49 (98%) | 0 | 14 (28%) | 36 (72%) | 0 |

The cell lines were cultured in the presence or absence of G418. For each ES clone HCVs were detected by FISH with a human alphoid-2 probe in 50 cells. The number (and percentage) of metaphase spreads showing respectively 0, 1, >1 and 2 HCVs are given. For the E10B1 cell line, 111 cells have been analysed.

The wording “cell line” means an embryonic stem cell line, “G418” means the antibiotic which is used for the selection of the recombinant HCV.

TABLE 2
Germline transmission of the HCV
A male ES cell line, carrying the HCV, was injected into the blastocyst of
C57BL/6 mice and implanted into a pseudopregnant female CD1 mouse. The
resulting male chimera 1 and 2 were crossed with female C57BL/6 mice and
the overall transmission to their offspring was measured (respectively 20 and
44% transmission). Five male F1 mice carrying the HCV and six female F1
mice carrying the HCV were crossed with respectively female and male
C57BL/6 mice. The overall male germline transmission to F2 was calculated
34% and the female germline transmission to F2 was 41%.

| Parent | Litters: HCV⁺/total | | | | | | | | % transmission |
|---|---|---|---|---|---|---|---|---|---|
| Chimera 1 (M) | 0/1 | 0/7 | 1/7 | 3/11 | 3/8 | 1/6 | | | 20 |
| Chimera 2 (M) | 0/2 | 0/5 | 5/7 | 4/7 | 6/13 | 3/5 | 2/8 | 5/10 | 44 |
| F1 male 1 | 1/10 | 1/2 | | | | | | | 34 |
| F1 male 2 | 6/8 | | | | | | | | |
| F1 male 3 | 1/8 | | | | | | | | |
| F1 male 4 | 2/6 | | | | | | | | |
| F1 male 5 | 1/1 | | | | | | | | |
| F1 female 1 | 6/9 | 7/13 | 3/9 | | | | | | 41 |
| F1 female 2 | 3/9 | | | | | | | | |
| F1 female 3 | 2/8 | | | | | | | | |
| F1 female 4 | 2/8 | | | | | | | | |
| F1 female 5 | 5/8 | | | | | | | | |
| F1 female 6 | 2/10 | | | | | | | | |

Tables 3A and 3B: Germ line transmission of HCV by F1 mice. Number HCV containing pups was analyzed by a PCR specific for the HCV on DNA of tail biopsies.

TABLE 3A
Seven male and and six female F1 mice (Chimera × C57B1/6)) carrying the
HCV were crossed with C57B1/6 mice. Tail fibroblasts of pups of subsequent
litters were analysed for the presence of the HCV by PCR. Overall transmission
was respectively 31% (male germline) and 36% (female germline).

| | | |
|---|---|---|
| | | % |
| | litters | germ line |
| parent | HCV⁺ pups/total pups | transmission |

1.1. MALE MEIOSIS (×C57B1/6)

| | | | | | | | | | |
|---|---|---|---|---|---|---|---|---|---|
| 1.1.1. (13) | 1/10 | 1/2 | 3/9 | 3/6 | 3/7 | | | | 31% |
| F1 m 1 | | | | | | | | | |
| (12) F1 m 2 | 6/8 | 2/5 | 1/9 | 1/8 | 5/10 | 4/9 | 4/4 | 1/3 | |
| (39) F1 m 3 | 1/8 | 0/10 | 1/1 | 0/5 | 0/3 | 0/7 | | | |
| (42) F1 m 4 | 2/6 | 2/7 | 3/6 | 0/1 | | | | | |
| (49) F1 m 5 | 1/1 | | | | | | | | |
| (51) F1 m 6 | 8/12 | 3/7 | 4/8 | 2/5 | 2/5 | 1/7 | 0/2 | | |
| (36) F1 m 7 | 0/1 | | | | | | | | |
| (44) F1 m 8 | 0/7 | 1/4 | 0/2 | 0/2 | 1/6 | 1/3 | 2/3 | 0/6 | |

1.2. FEMALE MEIOSIS (×C57B1/6)

| | | | | | | | | | | |
|---|---|---|---|---|---|---|---|---|---|---|
| 1.2.1. (14) | 6/9 | 7/13 | 3/9 | 4/7 | 6/14 | 5/15 | 7/13 | 6/17 | 2/5 | 36% |
| F1 v 1 | | | | | | | | | | |
| (53) F1 v 2 | 3/9 | 2/9 | 6/11 | 3/11 | 5/11 | 7/13 | 3/11 | 5/12 | 5/11 | |
| (54) F1 v 3 | 2/8 | 1/9 | 0/10 | 0/12 | 1/11 | 2/9 | 1/14 | | | |
| (57) F1 v 4 | 2/8 | 3/10 | 2/12 | 2/11 | 1/7 | 3/7 | 6/13 | 5/13 | | |
| (58) F1 v 5 | 5/8 | 6/11 | 5/12 | 4/13 | 6/12 | 6/11 | 6/11 | 2/8 | 2/7 | |
| (35) F1 v 6 | 2/10 | 2/10 | 6/11 | 4/10 | 3/11 | 6/11 | 8/8 | | | |
| (25) F1 v 7 | 1/7 | 2/9 | 5/11 | 2/2 | 2/6 | 5/12 | 3/8 | | | |

TABLE 3B
Seven male F1 mice (Chimera × C57B1/6, identical to the
animals used for the experiment described in table 3)
carrying the HCV were crossed with NMRI mice. Tail
fibroblasts of pups of subsequent litters were analysed
for the presence of the HCV by PCR. Overall transmission
was respectively 27% (male germline).

| | | |
|---|---|---|
| | | % germ |
| | litters | line trans- |
| Parent | HCV⁺ pups/total pups | mission |

1.3. MALE MEIOSIS (×NMRI)

| | | | | | | | |
|---|---|---|---|---|---|---|---|
| 1.3.1. (13) | 2/12 | 4/15 | 2/16 | 6/13 | 5/17 | 1/9 | 27% |
| F1 m 1 | | | | | | | |
| (12) F1 m 2 | 8/24* | 10/33* | 1/10 | 4/13 | 7/15 | 6/14 | |
| (39) F1 m 3 | 0/15 | 0/12 | 0/10 | 1/16 | 0/11 | 1/14 | |
| (42) F1 m 4 | 4/16 | 3/18 | 4/18 | | | | |
| (49) F1 m 5 | 5/13 | 1/2 | 5/11 | 6/16 | 1/1 | 2/14 | |
| (51) F1 m 6 | 7/13 | 2/11 | 8/17 | 3/15 | 5/13 | 4/13 | |
| (36) F1 m 7 | 5/13 | 5/16 | 8/16 | 4/18 | 3/11 | 4/12 | |
| (44) F1 m 8 | 2/13 | 5/10 | 5/15 | 5/16 | 4/11 | 4/16 | |

*two litters

TABLE 4
Neomycin gene expression from HCV.
The amount of HCV⁺ primary tail fibroblasts (analyzed
by FISH) and the percentage of G418 resistant colonies of
tail fibroblasts are depicted in bold.

| | FISH on tail | | | | |
|---|---|---|---|---|---|
| | fibroblasts | | | % G418 resistant fibroblasts | |
| | 0 | 1 | 2 | Number of | Number of |
| | HCV | HCV | HCV | clones − G418 | clones + G418 |
| 2. F1 mice | | | | | |
| Male 10 | 17% | 83% | 0% | 115 | 96 (83.4%) |
| Female 15 | 15% | 85% | 0% | 110 | 100 (90%) |
| 2.1.1.1. F2 mice | | | | | |
| Female 1 | 8% | 86% | 6% | 86 | 75 (87.2%) |
| Male 2 | 28% | 72% | 0% | 68 | 55 (80.8%) |
| Male 3 | 12% | 86% | 2% | 54 | 41 (75.9%) |
| Male 4 | 24% | 74% | 2% | 56 | 36 (64.3%) |
| Male 5 | 10% | 90% | 0% | 46 | 40 (86.9%) |

### REFERENCES

1. Robertson, G. et al. Position-dependent variegation of globin transgene expression in mice. Proc. Natl. Acad. Sci. U.S.A 92, 5371-5375 (1995).

2. Rivella, S. & Sadelain, M. Genetic treatment of severe hemoglobinopathies: the combat against transgene variegation and transgene silencing. Semin. Hematol. 35, 112-125 (1998).

3. Garrick, D., Fiering, S., Martin, D. I., & Whitelaw, E. Repeat-induced gene silencing in mammals. Nat. Genet. 18, 56-59 (1998).

4. Hashido, K. et al. Copy-dependent and position-independent expression of rat aldolase A gene. J. Biochem. (Tokyo) 118, 601-606 (1995).

5. Strauss, W. M. et al. Germ line transmission of a yeast artificial chromosome spanning the murine alpha 1(I) collagen locus. Science 259, 1904-1907 (1993).

6. Choi, T. K. et al. Transgenic mice containing a human heavy chain immunoglobulin gene fragment cloned in a yeast artificial chromosome. Nat. Genet. 4, 117-123 (1993).

7. This number left intentionally blank.

8. This number left intentionally blank.

9. Harrington, J. J., Van Bokkelen, G., Mays, R. W., Gustashaw, K., & Willard, H. F. Formation of de novo centromeres and construction of first-generation human artificial microchromosomes. Nature Genet. 15, 345-355 (1997).

10. Ikeno, M. et al. Construction of YAC-based mammalian artificial chromosomes. Nat. Biotechnol. 16, 431-439 (1998).

11. Henning, K. A. et al. Human artificial chromosomes generated by modification of a yeast artificial chromosome containing both human alpha satellite and single-copy DNA sequences. Proc. Natl. Acad. Sci. USA 96, 592-597 (1999).

12. Brown, K. E. et al. Dissecting the centromere of the human Y chromosome with cloned telomeric DNA. Hum. Mol. Genet. 3, 1227-1237 (1994).

13. Farr, C. J. et al. Generation of a human X-derived minichromosome using telomere-associated chromosome fragmentation. EMBO J. 14, 5444-5454 (1995).

14. Heller, R., Brown, K. E., Burgtorf, C., & Brown, W. R. A. Mini-chromosomes derived from the human Y chromosome by telomere directed chromosome breakage. Proc. Natl. Acad. Sci. USA 93, 7125-7130 (1996).

15. Mills, W., Critcher, R., Lee, C., & Farr, C. J. Generation of an 2.4 Mb human X centromere-based minichromosome by targeted telomere-associated chromosome fragmentation in DT40. Hum. Mol. Genet. 8, 751-761 (1999).

16. Handel, M. A., Cobb, J., & Eaker, S. What are the spermatocyte's requirements for successful meiotic division? J. Exp. Zool. 285, 243-250 (1999).

17. Hernandez, D., Mee, P. J., Martin, J. E., Tybulewicz, V. L. J., & Fisher, E. M. C. Transchromosomal mouse embryonic stem cell lines and chimeric mice that contain freely segregating segments of human chromosome 21. Hum. Mol. Genet. 8, 923-933 (1999).

18. Au, H. C., Mascarello, J. T., & Scheffler, I. E. Targeted integration of a dominant neo (R) marker into a 2- to 3-Mb human minichromosome and transfer between cells (In Process Citation). Cytogenet. Cell Genet. 86, 194-203 (1999).

19. Raimondi, E. et al. Gene targeting to the centromeric DNA of a human minichromosome. Hum. Gene Ther. 7, 1103-1109 (1996).

20. Guiducci, C. et al. Use of a human minichromosome as a cloning and expression vector for mammalian cells. Hum. Mol. Genet. 8, 1417-1424 (1999).

21. Vermeesch, J. R. et al. Multiple small accessory marker chromosomes (SACs) from different centromeric origin in a moderately mentally retarded male (1999).

22. Zhang, J. et al. Molecular analysis of the isochromosome 12P in the Pallister-Killian syndrome. Construction of a mouse-human hybrid cell line containing an i(12p) as the sole human chromosome. Hum. Genet. 83, 359-363 (1989).

23. Shen, M. H., Yang, J., Loupart, M. L., Smith, A., & Brown, W. Human mini-chromosomes in mouse embryonal stem cells. Hum. Mol. Genet. 6, 1375-1382 (1997).

24. Loupart, M. L., Shen, M. H., & Smith, A. Differential stability of a human mini-chromosome in mouse cell lines. Chromosoma 107, 255-259 (1998).

25. This number left intentionally blank.

26. Burgoyne, P. S. & Mahadevaiah, S. K. Unpaired sex chromosomes and gametogenetic failure in Chromosomes Today Volume 11 243-263 (1993).

27. Hunt, P., LeMaire, R., Embury, P., Sheean, L., & Mroz, K. Analysis of chromosome behavior in intact mammalian oocytes: monitoring the segregation of a univalent chromosome during female meiosis. Hum. Mol. Genet. 4, 2007-2012 (1995).

28. De Lange, T. Ending up with the right partner. Nature 392, 753-754 (1998).

29. Nimmo, E. R., Pidoux, A. L., Perry, P. E., & Allshire, R. C. Defective meiosis in telomere-silencing mutants of Schizosaccharomyces pombe. Nature 392, 825-828 (1998).

30. Cooper, J. P., Watanabe, Y., & Nurse, P. Fission yeast Taz1 protein is required for meiotic telomere clustering and recombination (see comments). Nature 392, 828-831 (1998).

31. Li, X. & Nicklas, R. B. Mitotic forces control a cell-cycle checkpoin. Nature 373, 630-632 (1995).

32. LeMaire-Adkins, R., Radke, K., & Hunt, P. A. Lack of checkpoint control at the metaphase/anaphase transition: a mechanism of meiotic nondisjunction in mammalian females. J. Cell Biol. 139, 1611-1619 (1997).

33. Parry, G. C., Erlich, J. H., Carmeliet, P., Luther, T., & Mackman, N. Low levels of tissue factor are compatible with development and hemostasis in mice. J. Clin. Invest 101, 560-569 (1998).

34. Drocourt, D., Calmels, T., Reynes, J. P., Baron, M., & Tiraby, G. Cassettes of the Streptoalloteichus hindustanus ble gene for transformation of lower and higher eukaryotes to phleomycin resistance. Nucleic Acids Res. 18, 4009 (1990).

35. Nagy, A., Rossant, J., Nagy, R., Abramow-Newerly, W., & Roder, J. C. Derivation of completely cell culture-derived mice from early-passage embryonic stem cells. Proc. Natl. Acad. Sci. U.S.A 90, 8424-8428 (1993).

36. Jeppesen, P., Mitchell, A., Turner, B., & Perry, P. Antibodies to defined histone epitopes reveal variations in chromatin conformation and underacetylation of centric heterochromatin in human metaphase chromosomes. Chromosoma 101, 322-332 (1992).

37. Kermouni, A. et al. The IL-9 receptor gene (IL9R): genomic structure, chromosomal localization in the pseudoautosomal region of the long arm of the sex chromosomes, and identification of IL9R pseudogenes at 9qter, 10pter, 16pter, and 18pter. Genomics 29, 371-382 (1995).

38. Vermeesch, J. R., Falzetti, D., Van Buggenhout, G., Fryns, J. P., & Marynen, P. Chromosome healing of constitutional chromosome deletions studied by microdissection. Cytogenet. Cell Genet. 81,68-72 (1998).

39. Shen, M. H., Mee, P. J., Nichols, J., Yang, J., Brook F., Gardner, R. L., Smith A. G. & W. R. A. Brown. A structurally defined mini-chromosome vector for the mouse germ line. Curr. Biol. 10, 31-34 (2000).

40. Shen, M. H. et al. Mammalian artificial chromosome ST1. Am. J. Hum. Genet. 65, A116 (2000)

41. Davis, B. R., Yannariello-Brown, J., Prokopishyn, N. L., Luo, Z. J., Smith, M. R., Wang, J., Carsrud, N. D. V., and Brown, D. B. (2000). Glass needle-mediated microinjection of macromolecules and transgenes into primary human blood stem/progenitor cells. Blood 95, 437-444.

What is claimed is:

1. A non-integrating chromosomal vector transmitted through male gametogenesis in each generation, said non-integrating chromosomal vector comprising:
an exogenous nucleic acid sequence.

2. The non-integrating chromosomal vector of claim 1 that
is further transmitted through mitosis in substantially all dividing cells, or
provides for a position independent expression of an exogenous nucleic acid sequence.

3. The non-integrating vector of claim 1 or claim 2 characterized in having a transmittal efficiency of at least 10% through each male and female gametogenesis.

4. The non-integrating chromosomal vector of claim 1, claim 2, or claim 3, wherein said gametogenesis occurs in a mammal.

5. The non-integrating chromosomal vector of claim 1, claim 2, claim 3, or claim 4, wherein said non-integrating chromosomal vector is circular.

6. The non-integrating chromosomal vector of claim 1, claim 2, claim 3, claim 4, or claim 5, wherein said non-integrating chromosomal vector is of human small accessory chromosome origin.

7. The non-integrating chromosomal vector of claim 1, claim 2, claim 3, claim 4, claim 5, or claim 6, wherein said non-integrating chromosomal vector comprises that deposited with the Belgian Coordinated Collections of Microorganisms—BCCM on Mar. 27, 2000 under accession number LMBP 5473 CB.

8. A process for producing a non-integrating chromosomal vector, said process comprising:
identifying a mitotically stable unit comprising an exogenous nucleic acid transmitted through male gametogenesis; and
introducing an entry site in said mitotically stable unit thus allowing for the integration of genes encoding a desired polypeptide, protein, ribozyme or anti-sense fragment of a gene into said unit.

9. An improved method of treating a disease in a subject of the type wherein the subject is administered a vector containing a nucleic acid of interest, the improvement comprising:
administering to the subject the non-integrating chromosomal vector of claim 1, claim 2, claim 3, claim 4, claim 5, claim 6, or claim 7 wherein, further, the exogenous nucleic acid sequence is the nucleic acid of interest.

10. A method of transfecting a cell of the type involving using a vector to transfect the cell, the improvement comprising:
using, as the vector to transfect the cell, the non-integrating chromosomal vector of claim 1, claim 2, claim 3, claim 4, claim 5, claim 6, or claim 7.

11. A method of expressing a nucleic acid sequence of interest in a cell, said method comprising:
transfecting a cell with the non-integrating chromosomal vector of claim 1, claim 2, claim 3, claim 4, claim 5, claim 6, or claim 7;
wherein the exogenous nucleic acid sequence of the non-integrating chromosomal vector is the nucleic acid of interest; and
expressing the exogenous nucleic acid sequence.

12. A process for producing a transgenic non-human organism, said process comprising:
transforming a non-human organism with the non-integrating chromosomal vector of claim 1, claim 2, claim 3, claim 4, claim 5, claim 6, or claim 7; and
producing a transgenic non-human organism.

13. A transfected cell, or transgenic non-human organism, said transfected cell or transgenic non-human organism comprising:
the non-integrating chromosomal vector of claim 1, claim 2, claim 3, claim 4, claim 5, claim 6, or claim 7, and
a biological unit selected from the group consisting of a cell, a plant, and a non-human animal having said vector therein.

14. A method of transfecting a cell of the type involving using a vector to transfect the cell, the improvement comprising:
using, as the vector, a vector comprising the small accessory chromosome or a nucleic acid corresponding to the human small accessory chromosome.

15. A recombinant vector comprising a nucleic acid derived from a small accessory chromosome together with a nucleic acid sequence of interest.

16. The recombinant vector of claim 15 wherein the nucleic acid sequence of interest has greater than about 10,000 nucleotides.

17. A non-integrating chromosomal vector having an attribute selected from the group of attributes consisting of
being transmitted through male gametogenesis in successive generations in a transgenic organism transformed with said non-integrating chromosomal vector;
being transmitted through mitosis in substantially all dividing cells having been transfected with said non-integrating chromosomal vector;
providing for a position independent expression of an exogenous nucleic acid sequence, and
any selection of said attributes,
wherein said non-integrating chromosomal vector is of small accessory chromosome origin and further comprises an exogenous nucleic acid sequence of interest.

18. A non-integrating chromosomal vector comprising:
a nucleic acid sequence of interest operably linked to means for transmission through male gametogenesis in each generation.

19. A non-integrating chromosomal vector comprising:
a nucleic acid sequence of interest operably linked to means for transmission through mitosis in substantially all dividing cells.

20. A non-integrating chromosomal vector comprising:
a nucleic acid sequence of interest operably linked to means for position independent expression of an exogenous DNA sequence.

21. A non-integrating chromosomal vector comprising:
a nucleic acid sequence of interest; and
a mitotically stable unit obtainable by a process comprising;
identifying a mitotically stable unit;
transfecting a cell containing said mitotically stable unit with at least one DNA molecule having a property selected from the group consisting of a selectable marker or a cloning site;
identifying integration of at least a part of said at least one DNA molecule into said mitotically stable unit.
